(19) United States
(12) Patent Application Publication
(10) Pub. No.: US 2022/0193282 A1
(43) Pub. Date: Jun. 23, 2022

(54) SYSTEM FOR MOBILE SMART DEVICES THAT STERILIZE TARGETS AND INCREASE THE TRANSPARENCY OF THE PROCESS

(71) Applicant: OMEC Medical Inc, San Jose, CA (US)

(72) Inventors: James Zheng, Fremont, CA (US); Daniel Cifelli, San Jose, CA (US); Anmin Zheng, Fremont, CA (US)

(21) Appl. No.: 17/558,047

(22) Filed: Dec. 21, 2021

Related U.S. Application Data
(60) Provisional application No. 63/130,068, filed on Dec. 23, 2020.

(51) Int. Cl. A61L 2/10; B08B 7/00; A61L 2/24; A61L 2/28

(52) U.S. Cl. CPC A61L 2/10; B08B 7/0057; A61L 2202/11; A61L 2/28; A61L 2202/14; A61L 2/24

(57) ABSTRACT

An apparatus for sterilizing a target includes an ultraviolet (UV) light source for emitting UV radiation for sterilizing the target, a measurement device for measuring a distance between the UV light source and the target, a controller for calculating a time duration for sterilizing the target in response to the measured distance, an attachment member for removably attaching an electronic device to the apparatus, and an interface circuit for electrically connecting the electronic device with the apparatus. The electronic device includes a processor configured to execute an application program and a display screen configured to display a result of the application program.

### CROSS-REFERENCES TO RELATED APPLICATIONS

This application claims priority to U.S. Provisional Patent Application No. 63/130,068, filed on Dec. 23, 2021, entitled “A System for Mobile Smart Devices that Sterilizes Targets and Increases the Transparency of the Process,” the disclosure of which is hereby incorporated by reference in its entirety for all purposes.

### BACKGROUND OF THE INVENTION

Ultraviolet (UV) irradiation is one of the most common methods for sterilization. While it is dangerous to human cells, it is equally effective at destroying harmful bacteria and other microorganisms. During the mid-twentieth century, the practice of using UV light to sanitize water was popularized and began to spread into other industries. In the modern day, UV lights are used everywhere, from meat packaging factories, air treatment facilities, water treatment facilities, and are seeing a rise in use for sterilizing operating rooms.

Because of the recent coronavirus outbreak, the general public has become concerned about sanitation and infection prevention. While procedures such as social distancing and masks can guard against person-to-person infection, the spread of the virus via contaminated surfaces has not been assuaged in a complete manner. As a result, mindful individuals hesitate to touch public surfaces and take great care to sanitize and disinfect objects in contact with others.

Thus, there is a need for a novel sterilization technique to improve the efficiency and transparency of the sterilization process.

### SUMMARY OF THE INVENTION

The present invention generally relates to the sterilization of pathogens using UV radiation, and more particularly to devices, systems, and methods of sterilizing an object with improved efficiency and transparency of the sterilization process.

Numerous benefits are achieved by way of the present disclosure over conventional devices and techniques. For example, embodiments of the present disclosure provide a sterilization system having a portable electronic device that is removably attached to the sterilization system via an attachment member. The electronic device can be a wireless handheld device or a mobile phone having an app configured to facilitate a user to operate the sterilization system. The app can be preinstalled or received wirelessly from a mobile network and includes a graphical user interface including one or more menus displayed on a touch panel display screen of the electronic device to guide a user through a plurality of sterilization processes with high efficiency and transparency.

In one embodiment, an apparatus for sterilizing a target includes an ultraviolet (UV) light source for emitting UV radiation for sterilizing the target, a measurement device for measuring a distance between the UV light source and the target, a controller for calculating a time duration for sterilizing the target in response to the measured distance, an attachment member for removably attaching an electronic device to the apparatus, and an interface circuit for electrically connecting the electronic device with the apparatus. The electronic device includes a processor configured to execute an application program and a display screen configured to display a result of the application program.

In one embodiment, a system for sterilizing a target includes an apparatus having an ultraviolet (UV) light source configured to generate an ultraviolet (UV) radiation, and a measurement device configured to measure a distance between the target and the UV light source. The system also includes an electronic device having an application program configured to calculate a dosage of the ultraviolet (UV) radiation for sterilizing the target, and an interface circuit configured to connect the electronic device with the apparatus and transfer information therebetween. The system can include a battery pack configured to provide power to the system and the total system weight including the battery pack can be less than 2.0 kg.

In one embodiment, a method for operating a sterilizing system is provided. The sterilizing system includes an apparatus containing a UV light source emitting a UV radiation for sterilizing a target and a measurement device configured to measure a distance between the UV light source and the target, and an electronic device containing a processor configured to execute an application program. The method includes measuring the distance between the UV light source and the target by the measurement device, calculating an activation time of the UV radiation in response to the measured distance, and activating the UV light source for the calculated activation time.

In an embodiment, the method also includes determining a tilt angle of the UV light source relative to a vertical direction perpendicular to a surface of the target and turning off the UV light source when the tilt angle exceeds a predetermined value. In some embodiments, the method additionally includes detecting a passage of a person or an animal using a sensor and turning off the UV light source upon detection of the passage of the person or the animal. In an exemplary embodiment, the method further includes selecting a dosage of the UV radiation by a user according to a type of pathogens and irradiating the target with the selected dosage of the UV radiation. The method may include emitting a light pulse by a visible light source toward the target and measuring a time of travel of the light pulse from the visible light source to the target and back from the target.

These and other embodiments of the disclosure, along with many of its advantages and features, are described in more detail in conjunction with the text below and corresponding figures.

### BRIEF DESCRIPTION OF THE DRAWINGS

FIG. 1 is a simplified block diagram of an apparatus for sterilizing a target according to an embodiment of the present disclosure.

FIG. 2A illustrates a front view of a system for sterilizing a target according to an embodiment of the present disclosure.

FIG. 2B illustrates a side view of the system of FIG. 2A.

FIG. 2C is a perspective view of a socket or base having a plurality of holes for receiving pins of an interchangeable UV light source according to an embodiment of the present disclosure.

FIG. 2D is a perspective view of an interchangeable UV light source according to an embodiment of the present disclosure.

FIG. 3A illustrates a front view of a system for sterilizing a target object according to an embodiment of the present disclosure.

FIG. 3B illustrates a rear view of the system of FIG. 3A.

FIG. 3C illustrates a side view of the system of FIG. 3A.

FIG. 4A illustrates a front view of a system for sterilizing a target object according to another embodiment of the present disclosure.

FIG. 4B illustrates a rear view of the system of FIG. 4A.

FIG. 4C illustrates a side view of the system of FIG. 4A.

FIG. 5A is Table 1 illustrating required dosages for killing pathogens under different conditions such as the tilt angle between a 280 nm UV radiation emitted from a UV light source and the vertical axis perpendicular to the target surface and distance of the UV light source and the target surface according to some embodiments of the present disclosure.

FIG. 5B is Table 2 illustrating required dosages for killing pathogens under different conditions such as the tilt angle between a 220 nm UV radiation emitted from a UV light source and the vertical axis perpendicular to the target surface and distance of the UV light source and the target surface according to some embodiments of the present disclosure.

FIG. 6A is a graph illustrating Coranavirus survival as a function of the dose of far-UVC (222 nm) light, plaque forming units PFUUV/PFU controls that may be used in embodiments of the present disclosure.

FIG. 6B is a graph illustrating that the far-UVC 222-nm light has a much shorter range in biological materials than the 254 nm light that may be used in embodiments of the present disclosure.

FIG. 6C is a plot illustrating a comparison of the wavelengths 254 nm vs. 222 nm for spore inactivation that may be used in embodiments of the present disclosure.

FIG. 6D is a plot illustrating a comparison of two major types of UVC-induced DNA damage (cyclobutane pyrimidine dimers (CPD) and 6,4-photoproducts).

FIG. 7, which includes FIGS. 7A and 7B, is a schematic flowchart illustrating operation steps of a sterilization system to sterilize a target according to an embodiment of the present disclosure.

FIG. 8 is a simplified flowchart of a method of a system for sterilizing a target object according to an embodiment of the present disclosure.

FIGS. 9A to 9C are photographs illustrating a backside view, a front side view, and a side view, respectively, of a sterilization system according to an embodiment of the present disclosure.

### DETAILED DESCRIPTION OF THE INVENTION

The present disclosure generally relates to the sterilization of pathogens using UV radiation. More specifically, the present disclosure relates to a handheld device that is operable to enable a user to determine the sterilization level of a target object and a method of operating the device.

One solution for sterilizing a surface using a robotic mobile platform with UV light is described in U.S. Pat. No. 8,779,391 to Flaherty, et. al. The robotic mobile platform can adjust time of exposure, distance from surface, and a sensor to detect infected areas. However, the volume and weight of the robotic mobile platform limit its applicability to sterilization of large areas of a flat surfaces (e.g., floors), and prevent it from being portable or wearable.

Other solutions include a stationary system for sterilizing a surface with UV light. For example, U.S. Pat. No. 10,092,669 to Marshall entails a UV radiation set-up for sterilizing door knobs and buttons that automatically irradiates after a person touches the specified object. Despite the automation, the user is not made clear as to whether the object has already been sterilized or how long ago it was last sterilized. In addition, the system is fixedly mounted to the door and is not portable or movable.

U.S. Pat. No. 10,603,394 to Farren, et. al. describes a portable UV sterilization device for the interior of objects and rooms, with a report generation system for sanitation cycles. However, the commonly occupied areas outdoors remain unprotected and many surfaces indoors are blocked by other objects. The sterilization device also proves to be impractical when needing to sterilize individual objects. U.S. Pat. No. 10,597,156 to Viel discloses a cleaning drone with UV light that measures the distance between itself and an object to move into appropriate range, yet the drone cannot fit into small spaces, may be hazardous in public areas, and may be banned in certain buildings. The drone may also be difficult to control and provides no indication on the level of sterilization. Thus, many UV sterilizing inventions include smart features, but they have narrow use range and limited applicability. Other commonly sold UV products such as UV wands suffer from a more fundamental problem, such as the strength of the light often is not enough to destroy harmful pathogens.

Many sterilization products using UV or UVC radiation are available. However, their performance is limited to the following factors:

a. The UVC products provide no information on whether a surface has become fully sterilized after any given amount of time.

b. The energy density of light decreases as the surface moves farther from the UV light source. UVC products do not have a simple way for users to know how close the product should be to the surface.

c. UVC light is invisible to the human eye. While some products have colored light to indicate when they are turned on, the products do not truly indicate what is the range of the UVC light. This can cause users to be uncertain as to what has been sterilized.

d. Some UVC products rely on batteries as a power source. The remaining power level in the batteries is not indicated, so that the products may suddenly run out of power while the user is outside a building and a power source is not available, which can be problematic for the user. As the level of a power supply is reduced, the UVC light flux may also decrease, thereby limiting sterilization capabilities.

e. Certain products have been proven to not sterilize properly. The products simply do not draw enough power to produce UV radiation strong enough to sterilize a surface in a reasonable amount of time.

f. Many UVC products are inconvenient to carry around due to their large size and/or weight, while some are simply intended to operate in a stationary location.

g. Some UVC products are not movable, which can be problematic when the UV light is blocked by certain objects or simply needs to be adjusted to reach certain areas.

Embodiments of the present disclosure provide improved sterilizing devices and methods that have several benefits over conventional devices and processes. In one exemplary embodiment, a sterilizing apparatus may connect to a mobile device and use the processing power of the mobile device to perform tasks such as calculating dosage and storing information. Objectives of the present disclosure provide a user with convenient and mobile sterilizing devices and processes to sterilize an object, along with giving the user control and information about the processes. In one embodiment, the mobile sterilizing device is portable or wearable by the user whenever the user goes outside or will come into contact with objects from the outside environment. When needing to sterilize a target object, the user can physically and electrically attach the mobile sterilizing device to their mobile phone and activate an “app,” while following the app's instructions on the display screen of the mobile phone. The user can move the mobile sterilizing device over the surface of the target object as much as necessary to sterilize the surface of the target object with UV radiation following the app's instructions. As used herein, an “app” is a piece of software that can be downloaded to a mobile phone from a wireless network and run on the mobile phone. The term “app” will also be referred to as an application program or a software program. The terms “target,” “target object,” “target area”, and “object” are interchangeably used. The term ultraviolet (UV) radiation may referred to UV radiation (wavelength range of 250 nm to 280 nm) and/or UVC radiation (wavelength range of 205 nm to 230 nm, e.g., 222 nm) where it is applicable.

One benefit of the present disclosure is shifting the burden of calculations and processing from the mobile sterilizing device onto a mobile phone that almost everyone carries in daily life. This way, the mobile sterilizing device can be made much lighter and easier to carry around, and require less power to operate. One other benefit is to utilize a unique downloadable app on the mobile phone. This way, the control interface can be designed to be easily used by all casual consumers, while simultaneously giving a great amount of control and specification to the mobile phone.

One embodiment of the present disclosure provides a sterilizing apparatus that includes a UVC light source for sterilization and a processor or controller to receive or deliver information, and an attachment member to removably attach the sterilizing apparatus to a mobile device (e.g., a mobile phone or a smart phone). The sterilizing apparatus also includes an interface circuit to transfer information between an app on the mobile device and the sterilizing apparatus. Embodiments of the present disclosure achieve several objectives.

A first objective is to efficiently sterilize a target by calculating the dosage and time duration needed to sterilize to a user-chosen percentage at a distance away from a sterilizing device. This can utilize a way to reliably measure the distance between a UV light source and the target. In one embodiment, the operation of measuring distance utilizes a laser diode emitting a laser light beam and disposed within the sterilizing device. An app (application program) would simply record the time it takes for the laser light beam to travel to and from the target, and the app would use the speed of light in air to calculate how far the laser light beam had traveled. The sterilizing device would also include a laser sensor to detect when the laser light beam bounces back from the target. In other embodiments, the operations of measuring a distance is performed on a mobile phone. The mobile phone could also emit a light beam and use the same process as above. However, because some mobile phone models do not include features (e.g., a laser diode) to emit a laser light beam, it would be more convenient that the sterilizing device contains the laser diode in this case.

After the distance between the UV light source and the target is known, the dosage can be calculated. For this, the emitted energy density of the UV light source will be used. In one embodiment, the sterilizing device has the type of UV light source stored as data, such that by connecting to the mobile phone and app, the information for emitted energy density can be automatically sent to the app and used for dosage calculations. In another embodiment, the user can enter this information into the app. The energy density of the sterilizing device can be listed on the sterilizing device. To prevent user confusion, some embodiments have this information automatically transferred. By knowing the distance, the energy density of the UV radiation after reaching the target can be calculated, which would produce a new value for the dosage. Thus, the amount of time needed to sterilize the target can be calculated and displayed to the user through the display screen of the mobile phone.

A second objective is to have a reliable process for connecting the sterilizing device to a mobile device (e.g., mobile phone) and relaying data (e.g., continually) between the sterilizing and mobile devices. In some embodiments, the means of connection would be via a Bluetooth communication. The sterilizing device would include a Bluetooth device to facilitate the communication process. In other embodiments, the sterilizing device would transfer information through a wire or cable attached to the sterilizing device. The sterilizing device's cable would connect to an adapter that accepts the various cable configurations of different mobile phones. As a result, the process enables all mobile phones of various types to reliably connect to the sterilizing device via a cable. In addition, the embodiments will have a means (e.g., an attachment member) to removably attach (mount) the sterilizing device to the mobile phone. In some embodiments, the attachment member can be a suction cup, a clip, a clamp, or others.

A third objective is to provide a system including a mobile device that has a display screen and control interface for a user to view and use the sterilizing device. This would be accomplished through an app on the connected mobile device. The app can be the control center of the whole system. The app can play a live-video feed of the background and place tabs over it. With these tabs, the user can access various settings and statistics. For example, the user could change the percentage of sterilization to achieve or set up a maximum sterilization time. The user could check how much battery power, phone or internal battery, is used per session of sterilization or see the records of previous sessions of sterilization. To begin the sterilization, a button icon would be placed on the (touch panel) display screen and upon being activated (pressed), the sterilizing device would begin measuring the distance between the UV light source and the target and display a visual indicator. The visual indicator can include text stating that a distance measurement has begun. If the user is too far away from the target for proper measuring, then more text would visually appear to try and direct the user for proper usage. After the distance has been measured, the app can display the estimated time for completing the sterilization, the percentage of sterilization that the user has selected, and a function button icon to be pressed to turn on the UV light source. Once the UV light source is turned on, the time until completion will count down and update if the distance changes. If the user frequently changes the distance, the visual indicator can appear again to direct the user. Furthermore, if the user intends to sterilize a large surface or object rather than a small to medium sized target, the app can measure the increased area of sterilization as the user moves the sterilizing device around. This increased area will be used to calculate the increased amount of time needed to sterilize to the desired percentage. Visual indicators can be displayed to direct how the user should be moving the sterilizing device around. After the sterilization is complete, a visual indicator can appear to indicate so, and the process will be recorded and stored on the app for later viewing.

A fourth objective is to provide safety features for both a user and other living creatures. A safety feature indicates the radiation range of the UV (or UVC) light. In one embodiment, the sterilizing device includes a visible light source (e.g., red LED, blue LED, green LED, a combinations thereof) to help indicate the radiation range of the UV light. The visible light would turn on concurrently with the UV light and be angled such that a light beam from the visible light would be directed to the same area as the UV light. In another embodiment, the app of the mobile device is configured to calculate the radiation range of the UV light and highlight the area being irradiated directly onto the video feed. Another technique for protection would be automatically turning off the UV light in front of a person or animal. In one embodiment, the sterilizing device includes an infrared sensor that would detect the sudden increase in infrared radiation from passing over a person or an animal. Upon sensing the passage of a person or animal, the sterilizing device turns off the UVC light source to prevent any possible harm to a person or animal. Other techniques for detecting a human or animal are also implemented in different embodiments, such as motion sensors and chemical sensors. In another embodiment, the sterilizing device operates under the assumption that the user would keep the mobile phone and the sterilizing device attached to it as horizontal as possible when sterilizing a target. If the mobile phone and the attached sterilizing device were to suddenly be turned upwards, the UVC light could shine on another person. Thus, the app could use the mobile phone's own accelerometers to measure sudden changes in angle and then automatically turn off the UVC light to prevent harm.

Embodiments of the present disclosure utilize various techniques for the sterilizing device to receive and store power. In one embodiment, the sterilizing device uses an external charger to charge a battery. The sterilizing device an also contain an internal battery that can be charged before usage. This can be accomplished through a charging case, where the charging case is charged beforehand and holds the sterilizing device in order to charge it before use. In another embodiment, the sterilizing device can have a separate or detachable power cord so that a user can directly charge the sterilizing device using a wall outlet before going outside with it. In other embodiments, the sterilizing device can use an electrical cable to connect to a mobile phone for transmitting information and for drawing power directly from the mobile phone. In this case, the sterilizing device does not need to have an internal battery and uses power from the attached mobile phone to power its components.

In one embodiment, the UVC light source is an UVC LED emitting light at the wavelength range of about 250-280, e.g., 270 nm. Any light source that can produce UVC light can work for the present disclosure, but the power consumption limits on mobile phone batteries is well suited for 270 nm UVC lights. The light source can be a semiconductor chip that directs all beams of light in a single direction, as opposed to light bulbs that randomly shine light in all directions. However, this wavelength of light is harmful for both humans and pets, so multiple safety features are required. In another embodiment, far UVC light (222 nm) can be used to avoid harming mammal cells. The UVC light sources can either be an excimer lamp or a microplasma light. In an embodiment, an external battery would be used to power the 222 nm light source. In this embodiment, the external battery could be placed in the pocket of a user and be connected with an electrical cable to the sterilizing device.

In one embodiment, the 222 nm light source is powered using a 110V adapter or a 12 Volt standard NiCd (metal hydride) battery pack. These are safer than lithium ion batteries. The output voltage for operating the 222 nm light source is about 6 kV (6,000 Volts DC).

The improved sterilization devices according to embodiments of the present disclosure have the following features:

a. The device can shine a UV light to destroy harmful pathogens while also being safe for human cells. This would allow the user to freely use the device anywhere without worrying about their own or others' safety.

b. The device can measure the distance between the UV light source and a surface in order to calculate how long the surface must be irradiated. After using the device for the calculated amount of time, the user can be certain that it is safe to touch the surface without being infected by a virus.

c. The device can display useful information such as time until sterilization is completed, how much power is left, and whether the user must adjust how the device is held. This would allow the user to be certain that the device is working properly.

d. The device can be easy to hold and easy to store, such that it can be brought or carried on-person to common public areas. The user can thus sterilize any commonly touched object that is usually avoided such as door handles or buttons.

e. The device can be easily charged or have its battery easily replaced.

f. The device can be easily moved around and reach narrow areas, allowing commonly difficult to reach areas to be sterilized.

g. The device can give users the choice to decide the percentage of biological contaminants neutralized, rather than having it pre-determined.

h. The device can be safely used with a built-in safety mechanism.

i. The device can have interchangeable UVC light sources (222 nm, 254 nm, 270 nm, etc.) pluggable into a base. The interchangeable UVC light sources can have a plurality of pins including one or more keyed guide pins for orienting and positioning the interchangeable UVC light sources in the base. The base can be a socket having a plurality of holes for receiving the plurality of pins.

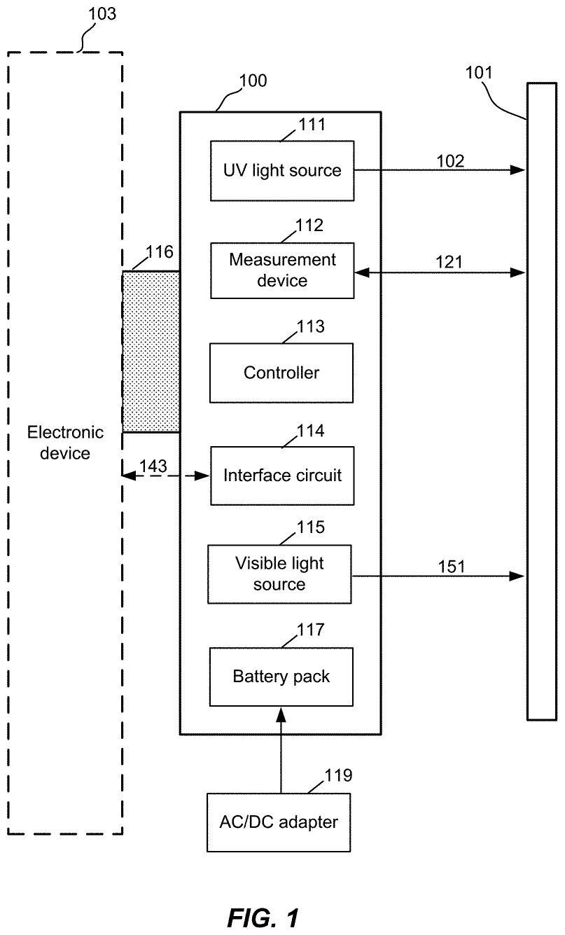

FIG. 1 is a simplified block diagram of an apparatus 100 for sterilizing a target according to an embodiment of the present disclosure. Referring to FIG. 1, apparatus 100 includes an ultraviolet (UV) light source 111 configured to radiate a target 101 with UV radiation 102, a measurement device 112 for measurement a distance 121 between the measurement device and the target, a controller 113 configured to calculate a time duration for the UV light source to be active to sterilize the target based on the measured distance, an interface circuit 114 configured to communicate data 143 to an external electronic device 103 and receive data 143 and/or control signals from the external electronic device to turn on and off the UV light source. Apparatus 100 may also include a visible light source 115 configured to emit a visible light beam 151 to target 101 and enable measurement device 112 to calculate the distance 121 based on the time of visible light beam 151 to travel to and from the target 101. Apparatus 100 may further include an attachment member 116 configured to mechanically and removably attach or mount external electronic device 103 to apparatus 100. In one embodiment, apparatus 100 also includes a battery pack 117 configured to provide power to the apparatus. In one embodiment, battery pack 117 is rechargeable. In one embodiment, battery pack 117 is removable and comprises, for example, a rechargeable NiCd-type battery pack. In one embodiment, apparatus 100 further includes an AC to DC (AC/DC) adapter 119 configured to generate a DC power from an AC outlet to charge battery pack 117. In one embodiment, AC/DC adapter 119 is external to apparatus 100. In one embodiment, UV light source 111 is interchangeable, i.e., a user can use a wide variety of UV light sources, such as LEDs (e.g., 254 nm, 270 nm, etc.) and/or lamps (e.g., 222 nm). UV light source 111 can have a plurality of pins including one or more keyed guide pins for orienting and positioning the interchangeable UVC light sources in a base or a socket (not shown). The base or socket can have a plurality of holes configured to receive the plurality of pins. Controller 113 can be operable to modify operations of apparatus 100 in response, at least in part, to a configuration of the plurality of pins.

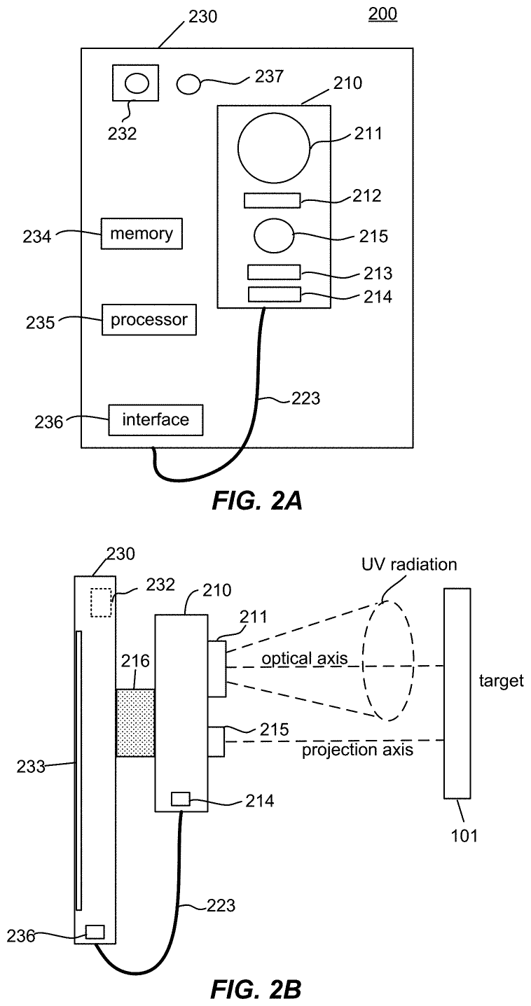

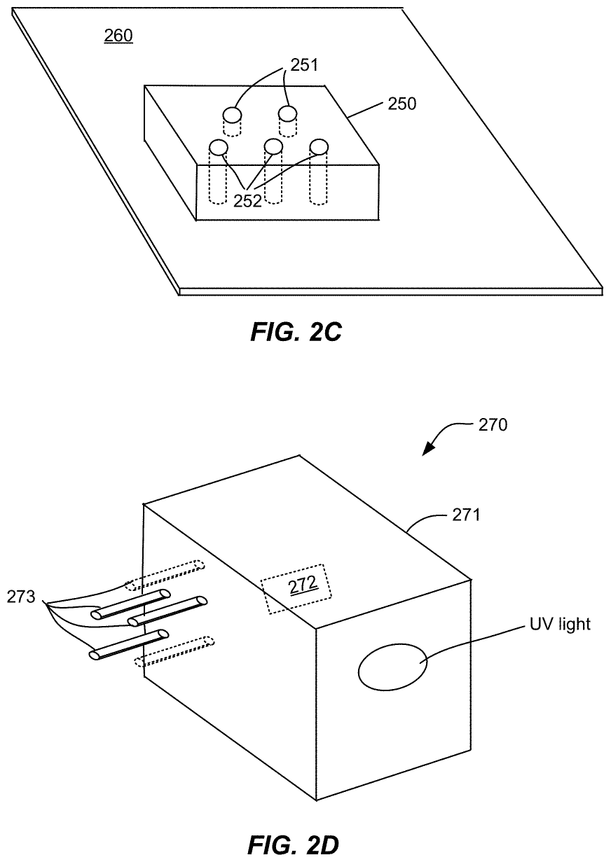

FIG. 2A illustrates a front view of a system 200 for sterilizing a target according to an embodiment of the present disclosure. FIG. 2B illustrates a side view of system 200 of FIG. 2A. Referring to FIG. 2A, system 200 includes a sterilizing apparatus 210 having a UV light source 211 configured to emit UV radiation to sterilize the target, a measurement device 212 configured to measure a distance between the sterilizing apparatus and the target, a controller 213 configured to calculate an activation time duration of the UV light source based on the measured distance and according to a user specified confidence level for the target sterilization, and an interface circuit 214 for communicating with an electronic device 230, which is mechanically and removably attached to sterilizing apparatus 210 through an attachment member 216. In one embodiment, sterilizing apparatus 210 includes a light source base (socket) having a plurality of holes for receiving a plurality of pins of UV light source 211. UV light source 211 can have one or more UVC LED devices or one or more UVC lamps. Controller 213 is operable to determine the type of UV light sources that is plugged into the light source base and modify operations of sterilizing apparatus 210 correspondingly. In one embodiment, sterilizing apparatus 210 further includes a visible light source 215 configured to emit a visible light beam for illuminating a surface of the target, and measurement device 212 configured to measure the time duration from the visible light beam traveling to the surface of the target and a reflected visible light beam back from the surface to calculate the distance between the surface of the target and the measurement device. The calculated distance is then transferred to electronic device 23 through interface circuit 214 for further processing and display to a user.

Electronic device 230 may include a digital camera 232 configured to take image data of the target to be sterilized or is being sterilized and a display screen 233 configured to display images taken by digital camera 232. Electronic device 230 may also include a memory device 234 configured to store the image data, a processor 235 configured to process the image data and display the processed image data to display screen 233, and an interface device 236 configured to communicate with interface circuit 214 of sterilizing apparatus 210. Electronic device 230 may further include an illumination light source 237. In some embodiments, sterilizing apparatus 210 may not include a visible light source so that system 200 utilizes illumination light source 237 to illuminate a surface of the target and measurement device 212 measures the distance between the system 200 and the target with illumination light source 237 of electronic device 230.

In some embodiments, electronic device 230 is electrically connected to sterilizing apparatus 210 through a wire 223. In one embodiment, wire 223 is a USB cable configured to provide power from electronic device 230 to sterilizing apparatus 210. In this case, each of sterilizing apparatus 210 and electronic device 230 includes a USB port configured to transfer data bidirectionally over the wire 223. In other embodiments, interface circuit 214 and interface device 236 are Bluetooth compatible devices configured to transfer data bidirectionally and wirelessly between sterilizing apparatus 210 and electronic device 230.

In one embodiment, electronic device 23 is a mobile phone. Attachment member 216 is a mobile phone holder having side clamps configured to hold electronic device 23, which is removably attached to sterilizing apparatus 210. In one embodiment, the projection axis of visible light source 215 is substantially parallel to the optical axis of UV light source 211. In one embodiment, the projection axis is separated from the optical axis and has an offset with the optical axis.

FIG. 2C is a perspective view of a socket 250, also referred to as a base, having a plurality of holes for receiving pins of an interchangeable UV light source according to an embodiment of the present disclosure. Referring to FIG. 2C, socket 250 may include a plurality of holes configured to receive pins of an interchangeable UV light source. Socket 250 may be mounted on a printed circuit board 260 of sterilizing apparatus 210. The plurality of holes may include holes 251 connected to a power source for providing power to an interchangeable UV light source (e.g., UV light source 211) and one or more keyed guide holes 252 for orienting and positioning the interchangeable UVC light source. In the example shown in FIG. 2C, two holes 251 are used for the power source and three holes 252 are used for the keyed guide holes. But it is understood that the number is arbitrary chosen for describing the example embodiment and should not be limiting. In the example shown, socket 250 is shown to have a rectangular shape, but it is understood that the socket can have non-rectangular shape, such as hexagonal, circular, oval, and other shapes.

FIG. 2D is a perspective view of an interchangeable UV light source 270 according to an embodiment of the present disclosure. Referring to FIG. 2D, interchangeable UV light source 270 includes a housing 271 for containing a UV light source 272 and a plurality of pins 273 connected to the housing. Some of the pins can be connectors for providing power to the UV light source, and one or more pins are keyed guide pins for indicating types and configurations of the interchangeable UV light source. In the example shown in FIG. 2D, the interchangeable UV light source is shown as having a plurality of pins, but it is understood that holes can also be utilized instead. In this case, the associated socket will have pins. In other words, the interchangeable UV light source and the socket can have complementary alignment features, the coupling between the interchangeable UV light source and the socket includes a plurality of male and female connectors. Controller 213 detects the position and/or the presence of the one or more keyed guide pins to determine the type and configuration of the interchangeable UV light source and adjust the operation of the sterilizing apparatus accordingly.

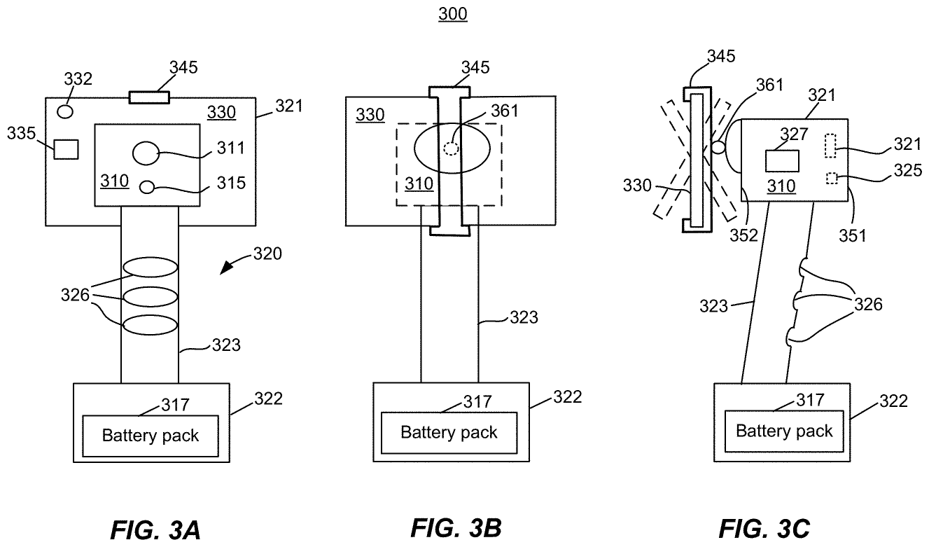

FIG. 3A illustrates a front view of a system 300 for sterilizing a target according to an embodiment of the present disclosure. FIG. 3B illustrates a rear view of system 300. FIG. 3C illustrates a side view of system 300. Referring to FIGS. 3A to 3C, system 300 includes a housing 320 having a top (upper) portion 321, a bottom (lower) portion 322, and a middle portion 323 connecting top portion 321 with bottom portion 322. In one embodiment, top portion 321 includes a sterilizing apparatus 310 mounted at a front 351 and an electronic device 330 mounted at a rear 352. Bottom portion 322 includes a cavity configured to contain a battery pack 317 for supplying power to sterilizing apparatus 310 and/or electronic device 330. Middle portion 323 may include a plurality of depressions 326 configured to accommodate fingers of a user, i.e., middle portion 323 serves as a handgrip for a user to hold system 300. That is, system 300 is a hand held sterilizing system or a portable sterilizing system. System 300 also includes an adjustable clamp 345 for holding electronic device 330. In one embodiment, adjustable clamp 345 may include two holding elements that are adjustable to hold electronic device 330 in a width direction (shown as a vertical direction). In one embodiment, electronic device 330 is a mobile phone having a length and a width that is shorter than the length, and the adjustable clamp 345 holds the mobile phone in the width direction.

System 300 further includes a ball joint pivot 361 coupled to adjustable clamp 345. Ball joint pivot 361 is coupled to top portion 321 of housing 320 and configured to enable the user to change an angular angle of electronic device 330 for viewing. That is, electronic device 330 may have an up and down tilt position as well as a left and right rotation. In one embodiment, adjustable clamp 345 and ball joint pivot 361 may be a part of an attachment member (e.g., attachment member 116 of FIG. 1, 216 of FIG. 2B). In one embodiment, system 300 does not have ball joint pivot 361, i.e., adjustable clamp 345 is fixedly connected to top portion 321 of housing 320, so that electronic device 330 is devoid of lateral and vertical movements.

In one embodiment, sterilizing apparatus 310 and electronic device 330 each may include components similar to those of sterilizing apparatus 210 and electronic device 230, respectively, of FIGS. 2A and 2B. For example, sterilizing apparatus 310 may include a UV light source 311 configured to emit UV radiation to sterilize a target, a measurement device (not shown) configured to measure a distance between the sterilizing apparatus and the target, a controller (not shown) configured to calculate an activation time duration of the UV light source based on the measured distance and according to a user specified confidence level for the target sterilization, and an interface circuit (not shown) for communicating with electronic device 330. In one embodiment, sterilizing apparatus 310 may also include a visible light source 315 configured to emit a visible light beam for illuminating a surface of the target and to enable the measurement device to calculate the distance between the target and the sterilizing apparatus.

In one embodiment, sterilizing apparatus 310 and electronic device 330 transfer data through a wireless communication protocol, e.g., Bluetooth communication protocol. In another embodiment, sterilizing apparatus 210 and electronic device 230 can have a bidirectional data transfer through a wire, with the exception that electronic device 230 does not provide power to sterilizing apparatus 210 through the wire because the power is provided by the built-in battery pack 317.

In one embodiment, battery pack 317 includes a NiCd rechargeable battery pack that can be charged remotely from system 300 and inserted into the bottom portion of housing 320 after being charged. In one embodiment, system 300 also includes a power management device (not shown) configured to generate voltages required for operations of system 300.

In one embodiment, electronic device 330 is a mobile phone that has its own power supply and charging system for the built-in lithium polymer or lithium ion battery. That is, the battery of electronic device 33 can be charged separated from the battery pack of system 3A.

In one embodiment, electronic device 330 may store an application (software) program (or “app”) and executes the application program using an embedded processor (e.g., processor 235) to calculate an activation time duration of the UV light source 311 based on a measured distance between sterilizing apparatus 310 and a target to be sterilized and/or according to a user specified confidence level. Electronic device 330 may update the stored “app” or replace the stored “app” by loading a new “app” from a wireless network.

In one embodiment, system 300 may further include a tilt angle measuring device 327 for detecting or calculating a tilt angle of the optical axis of the UV light source, tilt angle measuring device 327 is disposed at the top portion of housing 320 and configured to measure a tilt angle of the optical axis of the UV light source relative to the surface of the target. In an exemplary embodiment, visible light source 315 emits a circular shape light pattern on the planar surface of the target when the projection axis of the visible light source is substantially perpendicular to a planar surface of a target, and the projection axis is substantially parallel to the optical axis of the UV light source. Tilt angle measuring device 327 captures the reflected light pattern and calculates the tilt angle of the projection axis of the visible light source (i.e., the optical axis of the UV radiation) relative to a vertical axis of the planar surface based on an amount of distortion of the circular shape light pattern (according to a predetermined calculation formula). In one embodiment, tilt angle measuring device 327 is integrated in sterilizing apparatus 310. Sterilizing apparatus 310 communicates tilt angle data to electronic device 330, which displays the tilt angle data on a display screen and instruct a user to change the position of sterilizing apparatus 310 to reduce the tilt angle. Electronic device 330 can calculate the activation time duration of the UV light source in response to the received tilt angle data.

In one embodiment, electronic device 330 is mounted on top portion 321 of housing 320 and includes a digital camera 332 having an image sensor and a lens having an optical axis substantially parallel to the projection axis of visible light source 315. Electronic device 330 can utilize its digital camera 332 to capture the reflected light pattern and its processor 335 to calculate the tilt angle of the projection axis of the visible light source based on an amount of distortion of the circular shape light pattern and according to a predetermined calculation formula. Electronic device 330 then displays the results of the calculation on its display screen and instruct the user to change the position of sterilizing apparatus 310 to reduce the tilt angle.

In one embodiment, system 300 may further include a safety mechanism (not shown) configured to sense or detect a presence or passage of a person or a pet in the proximity of the UV irradiated area. The safety mechanism may include an infrared sensor configured to sense the proximity of a person or a pet. In one embodiment, the safety mechanism may include a motion sensor configured to sense or detect that a person or a pet moves into the UV radiation range. In one embodiment, the safety mechanism may include an accelerometer configured to detect a sudden lateral and/or vertical directional change of the UV radiation. When system 300 detects any one of these events, system 300 will turn off or deactivate the UV light source. In one embodiment, the safety mechanism is integrated in sterilizing apparatus 310. In one embodiment, the safety mechanism is an integrated circuit embedded in electronic device 330.

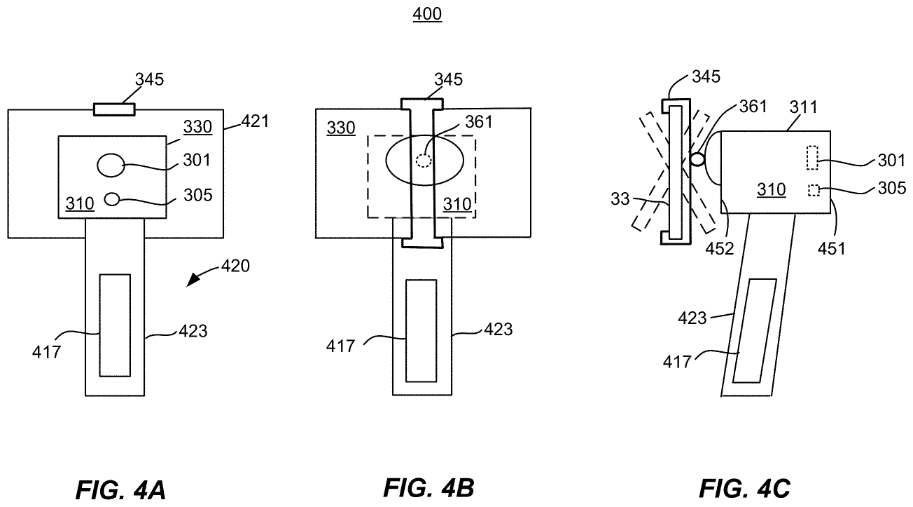

FIG. 4A illustrates a front view of a system 400 for sterilizing a target according to an embodiment of the present disclosure. FIG. 4B illustrates a rear view of system 400. FIG. 4C illustrates a side view of system 400. System 400 is similar to system 300 that has been described above, with the exception that housing 420 only has an upper portion 421 and a lower portion 423 coupled to the upper portion. Upper portion 421 includes sterilizing apparatus 310 mounted at a front 451 and an electronic device 330 mounted at a rear 452. Lower portion 423 includes a cavity configured to contain a battery pack 417 for supplying power to sterilizing apparatus 310 and electronic device 330. Lower portion 423 serves as a handgrip for a user to hold system 400. That is, system 400 is a hand held sterilizing system or a portable sterilizing system. In one embodiment, system 400 including a 12V battery pack 417 as a whole has a total weight less than 1 kg. In another embodiment, system 400 including a 18V battery pack 417 as a whole has a total weight less than 2 kg.

In one embodiment, system 400 may further include an AC-to-DC adapter that converts an AC power (110 V to 240 V AC) provided from a wall outlet to a DC power for charging battery pack 417 and for supplying DC voltages to sterilizing apparatus 310.

In one embodiment, system 400 may further include a safety mechanism (not shown) configured to sense or detect a presence of a person or a pet in the proximity of the UV irradiated area. The safety mechanism may include an infrared sensor configured to sense the proximity of a person or a pet. In one embodiment, the safety mechanism may include a motion sensor configured to sense or detect that a person or a pet moves into the UV radiation range. In one embodiment, the safety mechanism may include an accelerometer configured to detect a sudden lateral and/or vertical directional change of the UV radiation. When system 40 detects any one of these events, system 40 will turn off or deactivate the UV light source. In one embodiment, the safety mechanism (IR sensor, motion sensor, accelerometers) is integrated within sterilizing apparatus 310. In one embodiment, the safety mechanism is integrated within electronic device 330.

In one embodiment, data transfer between sterilizing apparatus 310 and electronic device 330 is through a USB connection. In one embodiment, data transfer between sterilizing apparatus 310 and electronic device 330 is through a Bluetooth communication.

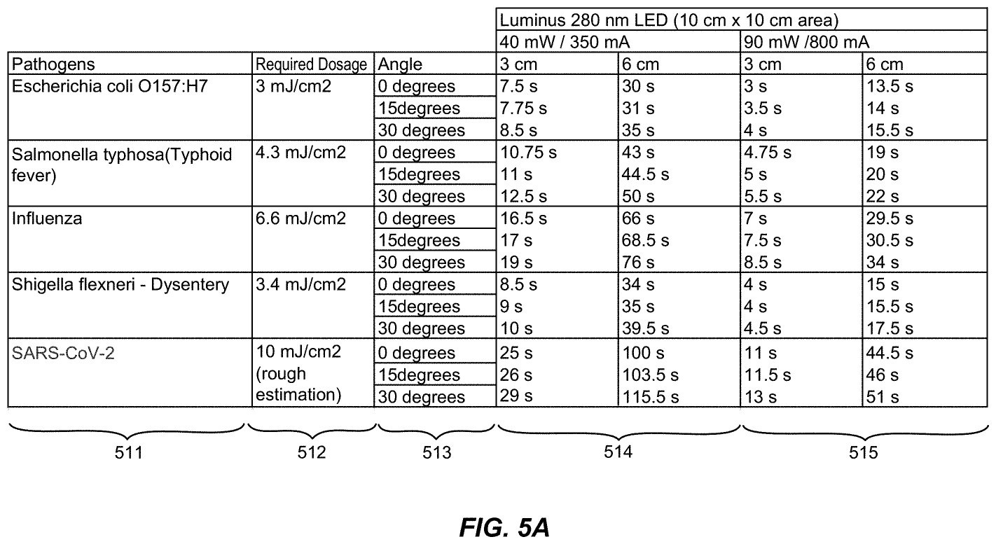

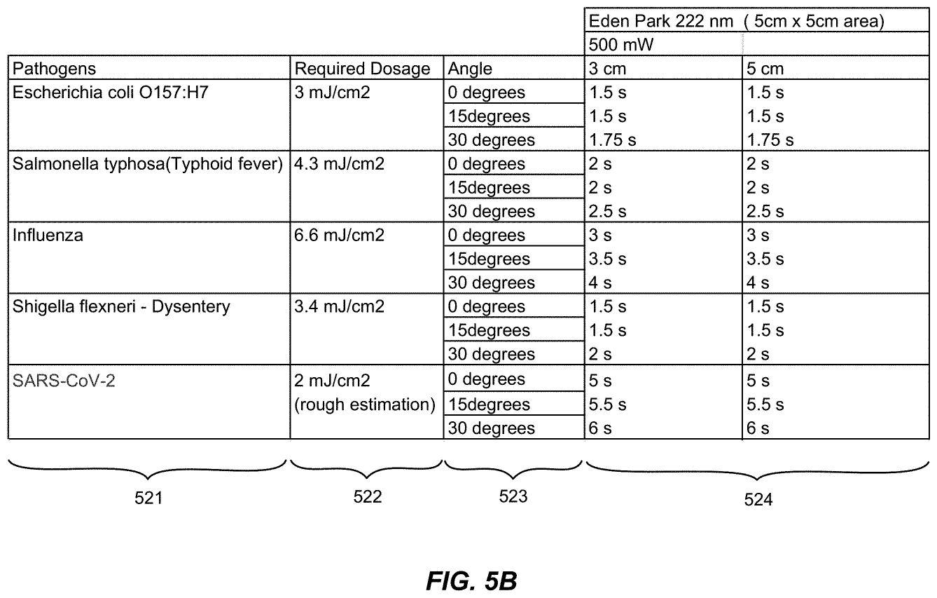

FIG. 5A is Table 1 illustrating required dosages for killing pathogens under different conditions such as the tilt angle between a 280 nm UV radiation emitted from a UV light source and the vertical axis perpendicular to the target surface and distances of the UV light source and the target surface according to some embodiments of the present disclosure. The following is a summary of the required dosages and activation time duration of the UV radiation shown in FIG. 5 as a function of the tilt angle and distance between a planar target surface and the optical axis of a 280 nm light source perpendicular to the planar target surface. The tilt angle refers to an angle between a real-world axis and a vertical axis perpendicular to a target surface, i.e., a tilt direction of the real-world axis of the UV light source relative to the vertical axis of the target surface. When the UV light source has a tilt angle, some of the UV radiation is closer and some is further away from the target. For example, a 10% reduction in flux/energy can occur with a tilt angle of about 30 degrees. An individual Luminus 280 nm LED is configured to emit UV light from a small area (e.g., 3 mm×3 mm) and can be considered as a point source, i.e., multiple LEDs can be arranged in various geometries or arrays to illuminate an area of 10 cm×10 cm and provide appropriate uniform illumination in close proximity to a target and not be considered a point source.

The left most block 511 in Table 1 lists the types of pathogens, block 512 lists the required dosages associated with the types of pathogens, block 513 lists the different inclination angle or tilt angle of the UV source relative to a vertical axis perpendicular to the target surface, block 514 has three columns associated with two distances (3 cm, 6 cm) of a UV light source with a wavelength 280 nm with a 40 mW power for a current of 350 mA and irradiating a 10 cm by 10 cm surface area and the activation time of the UV light source associated with the distances and the types of pathogens to be sterilized. Block 515 has three columns associated with two distances (3 cm, 6 cm) of a UV light source with a wavelength 280 nm with a 90 mW for a current of 800 mA and irradiating a 10 cm by 10 cm surface area and the activation time of the UV light source associated with the distances and the types of pathogens to be sterilized. It is noted that the tilt angle relative to the vertical axis is limited to 30 degrees because any tilt angle that exceeds this value would have the UV radiation shine on a person so that the built-in (integrated) safety mechanism would turn off the UV light source when the tilt angle of the optical axis of the UV light source is detected to be greater than 30 degrees.

As shown in Table 1, at close target distances the required sterilization time depends much more on the distance between the UV light source than the tilt angle. A high radiation power of the UV light source can reduce the required sterilization time significantly. As understood by those skilled in the art, the light energy from a point source goes with the square of the distance. In other words, for the same level of surface sterilization, the activation time duration of the UV light source quadruples of each doubling of the distance.

FIG. 5B is Table 2 illustrating required dosages for killing pathogens under different conditions such as the tilt angle between a 220 nm UV radiation emitted from a UV light source and the vertical axis perpendicular to the target surface and distance of the UV light source and the target surface according to some embodiments of the present disclosure. The following is a summary of the required dosages and activation time duration of the UV radiation shown in FIG. 5B as a function of the tilt angle and distance between a planar target surface and the optical axis of a 222 nm light source perpendicular to the planar target surface.

Referring to Table 2, left most block 521 in Table 2 lists the types of pathogens, block 522 lists the required dosages associated with the types of pathogens, block 523 lists the different inclination angles or tilt angles of the UV source relative to a vertical axis perpendicular to the target surface, and block 524 has two columns associated with two distances (3 cm, 5 cm) of a UVC light source with a wavelength 222 nm with a 500 mW power and irradiating a 5 cm by 5 cm surface area and the activation time of the UVC light source associated with the distances and the types of pathogens to be sterilized. The Eden Park 222 nm microplasma lamp device is not a point source at these close target distances because each of the more than 100 microplasma cells emits individually from inside the 5 cm×5 cm area. Hence, the “calculated” light fluxes and dosages in Table 2 do not fall off with the square of the distance when the light source is relatively close to the target. In the example shown, the target area has the same size as the emitting light spot size, so that anywhere with 5 cm of the emission area could produce the same light flux. Is it noted that the 222 nm microplasma lamp with a dosage of 2 mJ/cm² has a COVID-19 deactivation of greater than 99.9 percent vs. a 280 nm LED light source with a dosage of 10 mJ/cm². In other words, the 222 nm UVC lamp is about 5 times as effective as a UVC LED in the range from 270 nm to 280 nm. The 222 nm UVC lamp is about 10 times as effective as a UVC LED with a 254 nm wavelength from 270 nm.

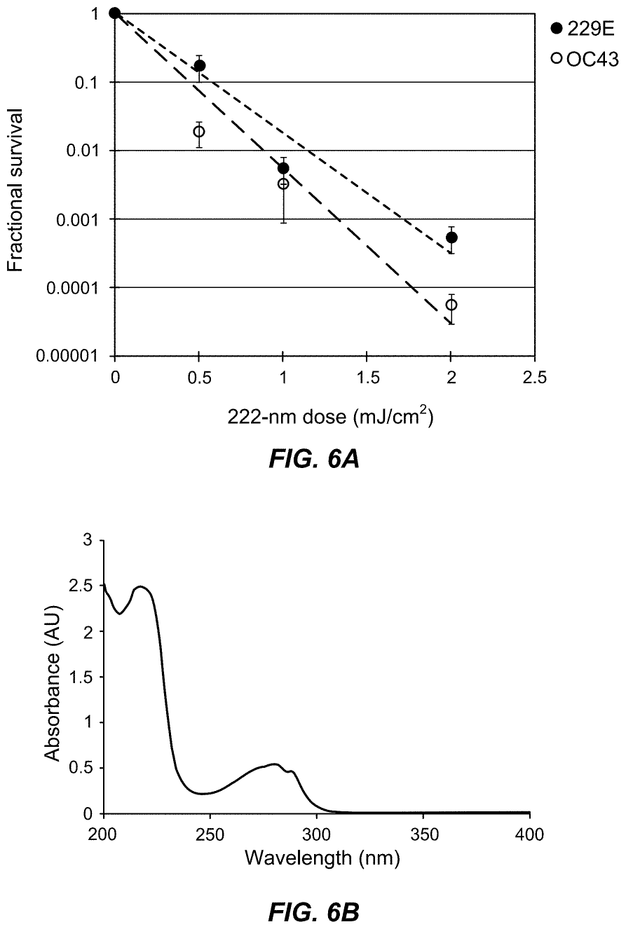

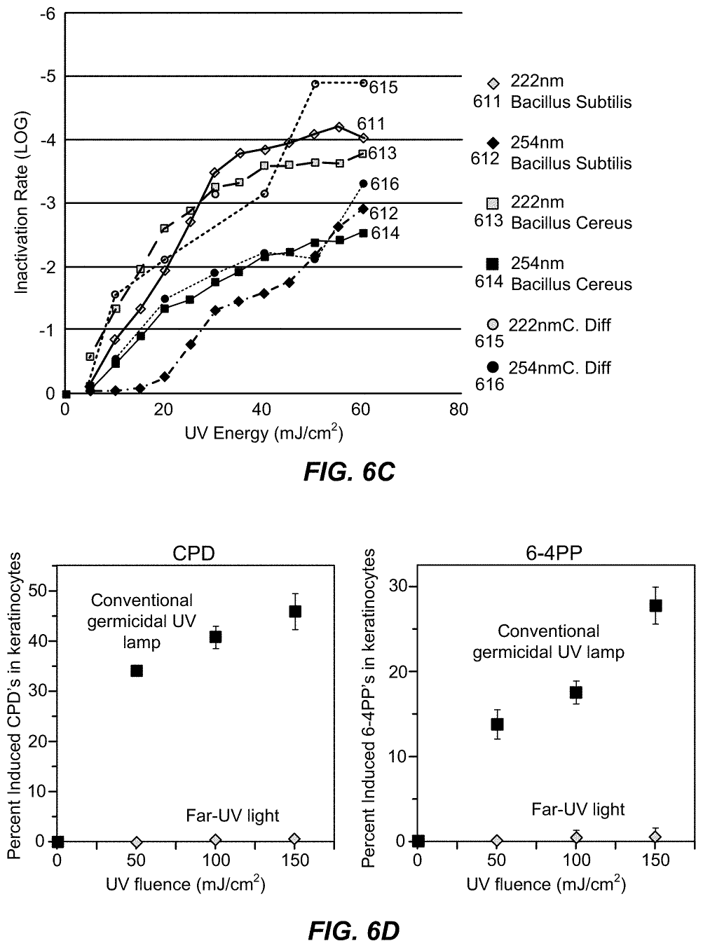

FIG. 6A is a graph illustrating Coranavirus survival as a function of the dose of far-UVC (222 nm) light, plaque forming units PFU_{UV}/PFU_{controls} that may be used in embodiments of the present disclosure. FIG. 6A is described in the publication “Far-UVC light (222 nm) Efficiently and Safely Inactivates Airborne Human Coronaviruses” by Manuela Buonanno et al., Scientific Reports, 2020 (http: www.nature.com/scientificreports). This paper is hereby incorporated by reference in its entirety. Referring to FIG. 6A, the x axis represents the 222-nm dose in mJ/cm², and the y axis represents the fractional survival in logarithmic scale. The filled (full) circle represents the alpha HCoV-229E virus and the unfilled (empty) circle represents the beta HCoV-OC43 virus. The graph shows the efficacy of the 222 nm light against the two airborne human coronaviruses. The lines represent the best-fit linear regressions for normalized logarithmic survival values as a function of the dose.

FIG. 6B is a graph illustrating that the far-UVC 222-nm light has a much shorter range in biological materials than the 254 nm light that may be used in embodiments of the present disclosure. Referring to FIG. 6B, the x-axis represents the wavelength in (nm), and the y-axis represents the absorbance in (AU) thereby illustrating the absorption spectrum of a peptide.

FIG. 6C is a plot illustrating a comparison of the wavelengths 254 nm vs. 222 nm for spore inactivation that may be used in embodiments of the present disclosure. The x-axis represents the UV energy in (mJ/cm2), and the y-axis represents the inactivation rate (i.e., fractional survival at a specific X-scale total dose); −1 LOG means 10% are still “active” or alive (or 90% inactivated/killed), −2 LOG means 1% are active (or 99% inactivated), −3 LOG means 0.1% are active (or 99.9% inactivated). Curve 611 represents the inactivation rate of Bacillus subtilis under the 222 nm wavelength. Curve 612 represents the inactivation rate of Bacillus subtilis under the 254 nm wavelength. Curve 613 represents the inactivation rate of Bacillus cereus under the 222 nm wavelength. Curve 614 represents the inactivation rate of Bacillus cereus under the 254 nm wavelength. Curve 615 represents the inactivation rate of Clostridium difficile under the 222 nm wavelength. Curve 616 represents the inactivation rate of Clostridium difficile under the 254 nm wavelength.

FIG. 6D is a plot illustrating a comparison of two major types of UVB-induced DNA damage with mercury vapor (e.g., UV black lights). The x axis represents the UV fluence or energy in (mJ/cm2), and the y axis represents the percent induced DNA damage. The plot on the left is for the cyclobutane pyrimide dimer (CPD) formation in human keratinocytes. Keratinocytes are the primary type of cell found in the epidermis, the outermost layer of the skin. In humans they constitute 90% of epidermal skin cells. The plot of the right represents the 6,4-photoproducts.

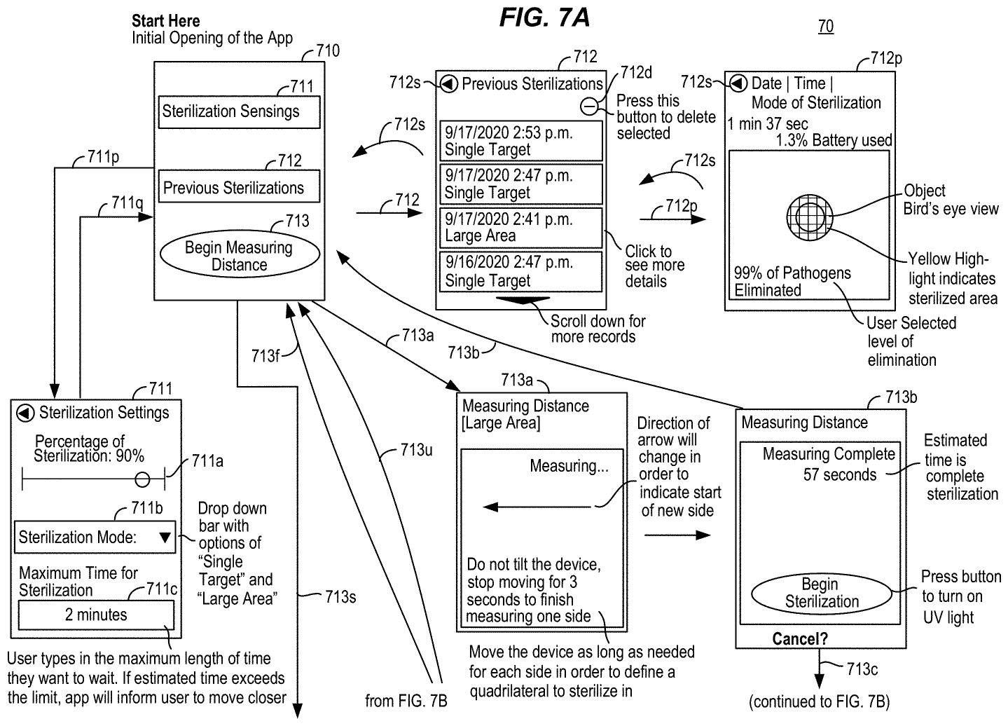

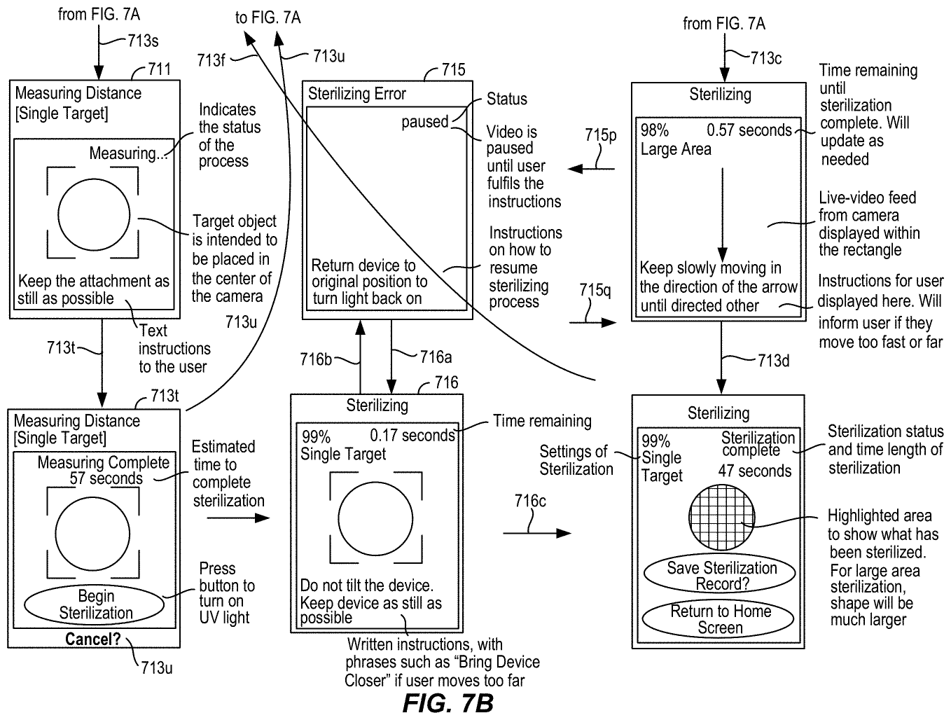

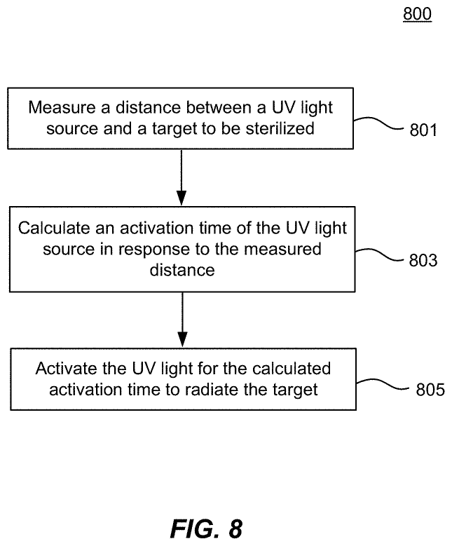

FIG. 7 including FIGS. 7A and 7B is an exemplary graph 700 illustrating operation steps of a sterilizing system for sterilizing a target according to an embodiment of the present disclosure. The sterilizing system may be a system including a sterilizing device coupled to an electronic device (e.g., the electronic device can be a mobile phone or a smart phone operable to receive an app from a telecommunications or wireless network and store the app in its memory) described in reference to FIG. 1 to FIG. 4A-C. Referring to FIG. 7A, the sterilizing system (hereinafter “the system”) starts by initializing an app stored in an electronic device (step 710). Step 710 may include a “sterilizing settings” operation 711, a “previous sterilizations” operation 712, and a “begin measuring distance” operation 713 shown on a display screen of the electronic device. Sterilizing settings in operation 711, when selected by the user (indicated by a bent arrow denoted 711p on the left-hand side) may include setting a percentage of sterilization desired by the user, e.g., 90% sterilization (settings 711a), a sterilization mode (setting 711b) from a drop down bar with options, e.g., single target or a large area, and/or a maximum time (setting 711c) for sterilization, when the maximum time is exceeded, the “app” of the system will inform the user to move the system closer to the target. When the sterilizing setting operation is complete (711q), operation 711 returns back to step 710.

“Previous Sterilizations” operation 712, when selected and viewed by the user, may include multiple previous sterilization records that the user can review and delete by pressing a “delete” button 712d. For example, a previous sterilization record 712p may show a bird's eye view of a sterilized area of an object that is indicated by a highlight color (e.g., yellow, green). In operation 712, the user may also delete any previously recorded sterilization data by pressing a “-” icon (712d). The user can go back to the original screen at step 710 by pressing the “<” icon (712s).

In “Begin Measuring Distance” operation 713 the user has the following options: (a) sterilization of a large surface area with the distance measurement process (713a). The display screen can indicate that the distance measurement operation is in progress. In one embodiment, the distance measurement process (713a) may further include measuring a surface area of the object to be sterilized and instruct the user to move the system in another direction to measure the surface area. The system also indicates the estimated sterilization time and the option whether to cancel or start the sterilization process (713b). When the user decides to process with the sterilization operation (713c), the system will indicate the time remaining and instruct the user to control the scan speed of the radiation beam, as shown in the sterilization operation (713c) in FIG. 7B. Referring to FIG. 7B, when the sterilization process is completed, the user has the option to save the sterilization record and return to the home screen of the system (713d). In one embodiment, the sterilization operation (713c) also provide the user the option to pause (715p) and resume the sterilization operation at a later time (715q). When the user resumes the sterilization operation, the system can provide instructions as the scan speed (shown in 713c) or place the UV light source closer to the object and maintain the tilt angle of the UV light source (716). The operation 716 may include multiple options, such as pause (716b), which proceeds back to operation (715), continue the sterilization operation (716c) and proceed to operation (713d), which has the option to save the sterilization record and return (713f) to the home screen (710) of the system.

In “Begin Measuring Distance” operation 713, the user can also select to sterilize a single target object (713s). In this operation, the single target object is displayed visually within the center of an image sensor of a digital camera (e.g., digital camera 232 of FIG. 2A). The screen also displays text instructions to the user (e.g., keep the system including the UV light source as still as possible. The app then proceeds to operation 713t which indicates to the user the estimated sterilization time of the target object. In operation 713t, the user has the options to either cancel (713u) the sterilization and the system return back to the home screen in initial step 710 or operation 716 to sterilize the target object. Operation 716 can also provide text instructions to the user, e.g., “bring the system close to the target object,” “do not tilt the system,” or “keep the system still,” etc. When the sterilization of the single target object is complete, the sterilization operation proceeds to operation 713d, where the user can save the sterilization record and return to the home screen 710.

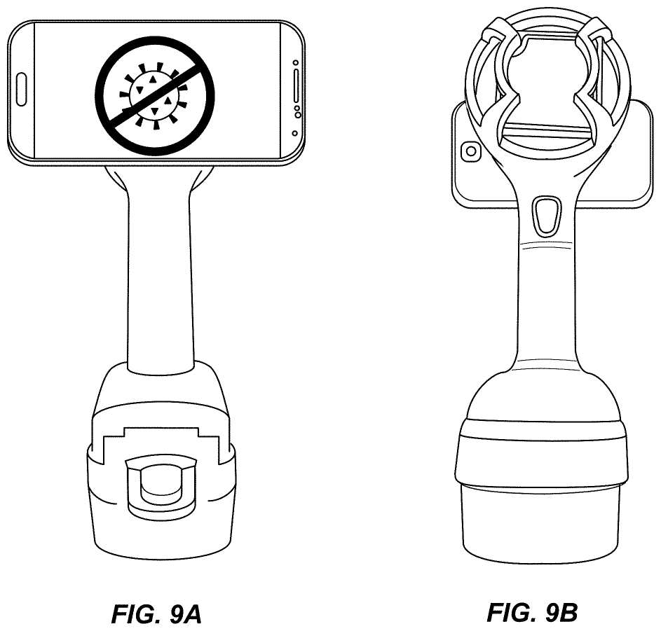

FIG. 8 is a simplified flowchart of a method 800 of a system for sterilizing a target according to an embodiment of the present disclosure. The system may include a sterilizing apparatus coupled to an electronic device. The system may be a system 20, 30, or 40 of FIG. 2, 3, or 4. The sterilizing apparatus may be the sterilizing apparatus 100 of FIG. 1 or sterilizing apparatus 210 of FIGS. 2A and 2B. In one embodiment, the sterilizing device include a UV light source configured to emit a UV radiation to sterilize the target, a measurement device configured to measure a distance between the sterilizing apparatus and the target, a controller configured to calculate an activation time of the UV light source based on the measured distance, and an interface circuit configured to communicate with the electronic device. The electronic device includes a digital camera configured to take image data of the target to be sterilized or is being sterilized, a display screen configured to display images taken by the digital camera, a memory device configured to store sterilization records of the sterilized target, an antenna configured to receive an app from a mobile network, and a processor configured to execute the received app and display instructions to a user on the display screen to operate the system, and an interface device configured to communicate with the sterilizing apparatus. Method 80 includes measuring the distance between the UV light source and the target at block 801, calculate an activation time of the UV light source in response to the measured distance at block 803, and activate the UV light to radiate the target based on the calculated activation time at block 805.

In one embodiment, the distance measurement is performed by shining a visible light beam at the surface of the target and measures the amount of time that the visible light beam travel to the target and bounce back. By knowing the speed of light in air, the time travelled can be used to calculate the length of the distance. In one embodiment, the UV light source includes an excimer or microplasma lamp, and the wavelength of light can vary from 207 nm to 280 nm. Knowing both the wavelength of light and power consumed, the system can calculate the energy density of the emitted light and how it would change as the UV light travels the distance to the target. With the energy density at the surface known, the system can now calculate how much time is needed to sterilize the surface for any confidence level set by the user. In one embodiment, the system continuously measures the distance between the UV light source and the target and adjusts the activation time of the UV light source in response to the changes in distance until the sterilization reaches a user predetermined confidence level. The target can be any commonly touched objects, such as doorknobs or grocery products picked up from a store.

In one embodiment, the measurement device of the sterilizing apparatus includes a light generator for emitting a light beam to the target and a light sensor for receiving a portion of the light beam reflected from the target. The measurement device outputs a distance measurement signal indicating a measured distance, which the sterilizing apparatus transfers to the electronic device for display. Other relevant information such as the estimated time until the sterilization is completed, real-time update notification to the user when the distance has been changed by the user and other useful information (amount of remaining power, percentage of sterilization) can also be displayed by the electronic device. In one embodiment, the data transfer between the sterilizing apparatus and the electronic device is over an electrical cable (e.g., USB connection). In one embodiment, the data transfer between the sterilizing apparatus and the electronic device is over a wireless communication link (e.g., Bluetooth communication). In one embodiment, the system includes an attachment member that removably attaches the electronic device to the sterilizing apparatus.

In one embodiment, the display screen of the electronic device includes a touch control panel display on which a plurality of menu options are displayed. The exemplary menu options are shown in FIGS. 7A and 7B (e.g., “Sterilization Settings,” “Previous Sterilization,” “Begin Measuring Distance” in step 710, submenu option 711 (“Sterilization Level,” “Sterilization mode,” “Maximum Time for Sterilization”), submenu option 712 (Records of Previous Sterilizations), submenu option 713 (“Measuring Distance”, “Sterilizing Error,” “operation instructions to the user,” etc.). In one embodiment, the sterilizing setting in submenu option 711 may facilitate the user to select the sterilization time based on the UV light source, e.g., a 280 nm UV light of Luminus, or 222 nm UV light of Eden Park Illumination, Inc. In one embodiment, the app is operable to save sterilization data so that the user can review past sterilization events.

In one embodiment, the sterilizing apparatus also includes a visible light source operable as a visual indicator to the user for what is being sterilized. The visible light source can have a projection axis that is aligned with the optical axis of the UV light source.

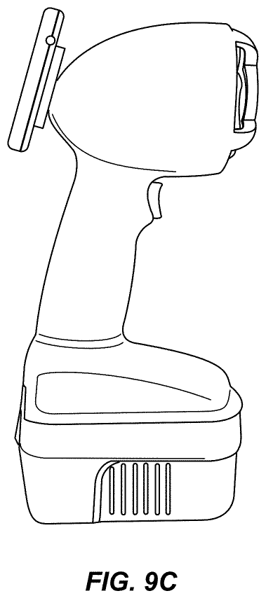

FIGS. 9A to 9C are photographs illustrating a backside view, a front side view and a side view, respectively, of a sterilization system according to an embodiment of the present disclosure. Referring to FIG. 9A, an electronic device (i.e., a mobile phone) is mounted on the back side of the sterilization system, the electronic device includes a touch panel display screen operable to display an operation menu to a user for operating the system. Referring to FIG. 9B, a sterilization apparatus is mounted on the front side and includes a UV light source emitting a UV radiation for irradiating an object to be sterilized. Referring to FIG. 9C, the system includes an upper portion having the UV light source mounted at the front, the electronic device mounted at the back, a lower portion for storing a battery pack, and a middle portion connecting the upper and the lower portions. It is noted that the system has an appearance of a cordless drill with a battery pack, it is understood that the photographs are one illustrative embodiment and not limiting, one of skill in the art will appreciate that various forms, shapes, and sizes of the system are possible.

The embodiments above have been described in relation to a system including a handheld sterilizing apparatus and a handheld electronic device that is removably attached to the sterilizing apparatus. The system includes a UV (250 nm to 280 nm) or UVC (222 nm) light source for the irradiation of an object with UV or UVC radiation. The system also includes a battery pack (e.g., a NiCad rechargeable battery pack) or a power adapter and power electronics for generating necessary powers for the operation of the UV or UVC light source. The system further includes a safety mechanism configured to power off the UV or UVC light source when the system detects that the UV or UVC light source has an inclination angle or tilt angle exceeding a predetermined value such that the target will not be irradiated by the UV or UVC radiation.

What is claimed is:

1. An apparatus for sterilizing a target, the apparatus comprising:
an ultraviolet (UV) light source configured to emit UV radiation for sterilizing the target;
a measurement device configured to measure a distance between the UV light source and a target;
a controller configured to calculate a time duration for sterilizing the target in response to the measured distance;
an attachment member configured to removably attach an electronic device to the apparatus, the electronic device comprising a processor configured to execute an application program and a display screen configured to display a result of the application program; and
an interface circuit configured to electrically connect the electronic device with the apparatus.

2. The apparatus of claim 1, further comprising:
a housing;
a battery pack disposed in the housing; and
an AC/DC adapter disposed in the housing and configured to generate a DC power from an AC outlet to charge the battery pack.

3. The apparatus of claim 2, wherein the battery pack comprises a NiCd-type rechargeable battery pack.

4. The apparatus of claim 1, wherein the UV radiation comprises a wavelength range between 220 nm and 280 nm.

5. The apparatus of claim 1, further comprising:
a safety mechanism configured to determine a tilt angle of the UV light source relative to a vertical direction perpendicular to a surface of the target and turn off the UV light source when the determined tilt angle exceeds a predetermined value.

6. The apparatus of claim 5, wherein the safety mechanism comprises an accelerometer configured to measure a change of the tilt angle of the UV light source.

7. The apparatus of claim 1, further comprising an infrared sensor configured to detect a passage of a person or an animal and turn off the UV light source when the passage of the person or the animal has been detected.

8. The apparatus of claim 1, wherein the electronic device is a smart phone.

9. The apparatus of claim 1, wherein the UV light source comprises an ultraviolet-C (UVC) laser diode operable at a voltage level in a range between 2000 volts and 6500 volts.

10. The apparatus of claim 1, further comprising a visible light source configured to emit a visible light pulse to the target, wherein the measurement device is configured to determine a time of travel from the visible light pulse to the target and reflected back from the target.

11. The apparatus of claim 1, further comprising an indicator configured to indicate an operation state of the UV light source.

12. The apparatus of claim 1, wherein the interface circuit comprises a Bluetooth protocol.

13. The apparatus of claim 1, further comprising a socket having a plurality of holes configured to receive a plurality of pins of the UV light source, wherein the controller is operable to determine a configuration of the plurality of pins and modify operations of the apparatus in response to the configuration of the plurality of pins.

14. The apparatus of claim 13, wherein the plurality of pins comprises one or more keyed guide pins for orienting and positioning the UV light source.

15. A system for sterilizing a target, the system comprising:
an apparatus comprising an ultraviolet (UV) light source configured to generate an ultraviolet (UV) radiation and a measurement device configured to measure a distance between the target and the UV light source;
an electronic device including an application program configured to calculate a dosage of the ultraviolet (UV) radiation for sterilizing the target; and
an interface circuit configured to connect the electronic device with the apparatus and transfer information therebetween.

16. The system of claim 15, further comprising:
a housing configured to enclose the apparatus, the housing comprising a front side facing toward the target, and a back side facing away from the target, wherein the electronic device is attached on the back side.

17. The system of claim 16, wherein the electronic device comprises a processor configured to execute the application program and a display screen facing away from the target and configured to display a result of the executed application program.

18. The system of claim 16, further comprising:
an attachment member; and
a rechargeable battery pack,
wherein the attachment member is disposed at a top portion of the housing and configured to removably attach the electronic device to the back side, and
wherein the rechargeable battery pack is enclosed in a bottom portion of the housing.

19. The system of claim 15, further comprising a safety mechanism configured to determine an inclination angle of the UV light source and turn off the UV light source when the determined inclination angle exceeds a predetermined value.

20. The system of claim 15, further comprising an infrared sensor configured to detect proximity of a person or an animal and turn off the UV light source when the proximity of the person or the animal has been detected.
